(19) United States
(12) Patent Application Publication
(10) Pub. No.: US 2023/0233826 A1
(43) Pub. Date: Jul. 27, 2023

(54) TATTOO INK DISPENSER

(71) Applicant: Anastacio Castillo, Taylorsville, UT (US)

(72) Inventor: Anastacio Castillo, Taylorsville, UT (US)

(21) Appl. No.: 18/100,799

(22) Filed: Jan. 24, 2023

Related U.S. Application Data
(60) Provisional application No. 63/302,497, filed on Jan. 24, 2022.

(51) Int. Cl. A61M 37/00

(52) U.S. Cl. CPC A61M 37/0084

(57) ABSTRACT

A tattoo ink dispenser includes a tube. The tube includes at least a removable top and a center compartment. The center compartment is a hollow structure that stores ink. The tube further includes a bottom portion of the tube with a central opening to place and hold a needle therein. The stored ink in the center compartment flows downwards and adheres to the needle to create a tattoo on a surface.

### RELATED APPLICATIONS

The present application claims the benefit under 35 U.S.C. § 119(e) of U.S. Provisional Pat. Application No. 63/302,497 filed Jan. 24, 2022, which is incorporated be reference herein.

### TECHNICAL FIELD

The present disclosure generally relates to a tattoo machine, and more specifically relates to apparatuses and methods for a tattoo ink dispenser.

### BACKGROUND

Tattoos have become increasingly common among the general population. Getting a tattoo typically involves a lengthy and meticulous process of placing ink below the surface of a person’s skin using a tattoo machine. Tattoo machines conventionally require tattoo artists to manually dip a needle into a separate container of ink throughout the tattooing process. This can often add unnecessary time and effort to an already extensive process.

### BRIEF DESCRIPTION OF THE DRAWINGS

The present disclosure will be understood more fully from the detailed description given below and from the accompanying drawings of various implementations of the disclosure.

FIG. 1 illustrates a perspective view of a tattoo ink dispenser, in accordance with some embodiments of the present disclosure.

FIG. 2 illustrates an aerial top view of a tattoo ink dispenser, in accordance with some embodiments of the present disclosure.

FIG. 3 illustrates a perspective view of a tattoo ink dispenser that can be fitted with an interchangeable ink cartridge, in accordance with some embodiments of the present disclosure.

FIGS. 4A-B illustrate a perspective view of a removable tattoo ink cartridge, in accordance with some embodiments of the present disclosure.

### DETAILED DESCRIPTION

Aspects of the present disclosure are directed to a tattoo ink dispenser. A conventional tattoo machine typically includes a cylindrical or tube-like structure containing a needle within the structure, where the needle protrudes some distance beyond the tip of the tube. The conventional tattoo machine does not include ink (also referred to as pigment) within the tattoo machine. Instead, tattoo artists need to manually dip the needle of the tattoo machine into a separate container of ink. The tattoo machine then creates an up-and-down motion of the needle that allows the tattoo artist to place the ink by penetrating a person’s skin to a layer beneath the surface. Due to the amount of ink that tattooing typically requires, especially large tattoos, tattoo artists will need to dip the needle into the separate container of ink numerous times throughout the tattooing process.

The need to pause tattooing and remove the needle from beneath a person’s skin each time the tattoo artist has to get more ink can consume a large and unnecessary amount of time. This can be inefficient for tattoo artists who would prefer to reduce the amount of time they need to spend on each tattoo in order to have time to accept more tattoo customers and do more tattoos. Further, tattoo customers who typically experience pain while getting tattooed would prefer a tattooing process that takes less time. Additionally, having to dip the needle into ink numerous times throughout the tattooing process can be disruptive to tattoo artists who need to be concentrated and precise as they place the needle with ink below the surface of someone’s skin.

Aspects of the present disclosure overcome the above-noted and other deficiencies of conventional tattoo machines by providing a tattoo ink dispenser that stores pigment (e.g., ink) within the tattoo ink dispenser, thus eliminating the need for tattoo artists to dip the needle into a separate container of ink. In some embodiments, the tattoo ink dispenser can include a tube-like structure (hereinafter referred to as a “tube”). The tube can include a removable top (e.g., a cap), a center compartment, an ink reservoir, and a needle. The tattoo ink dispenser can also include an exterior grip surrounding the tube that allows a tattoo artist to hold the tattoo ink dispenser. A tattoo artist can fill in a specific amount of ink that they would like to use for the tattoo into the center compartment of the tattoo ink dispenser by removing the removable top and filling the center compartment with the ink.

As the tattoo artist holds the tattoo ink dispenser and begins to create tattoo pattern on a body part of a person, the ink filled in the center compartment can flow downwards towards the ink reservoir connected at one end (e.g., the bottom) of the center compartment. The ink reservoir can be configured to extend alongside an outer surface (e.g., an enclosure, also referred to as an “enclosing” in the figures) surrounding the needle. The enclosing can have a small opening at one of its ends (i.e., the end closest to the tip of the tube). The ink can flow downwards from the center through the ink reservoir into the small opening of the enclosing, where the ink then adheres itself to the needle. As the ink flows downward, air pressure will flow upward, thus keeping the ink from over-dispensing onto the needle and/or skin. Because the design of the tattoo ink dispenser allows for ink to already be contained within the tattoo ink dispenser, tattoo artists no longer need to manually dip the tattoo machine into a separate container of ink throughout the tattooing process. Instead, the tattoo artist simply needs to fill the desired amount of ink into the tattoo ink dispenser and begin tattooing. This thus results in a significant reduction in time spent on the tattooing process, benefitting both the tattoo artist and tattoo customer.

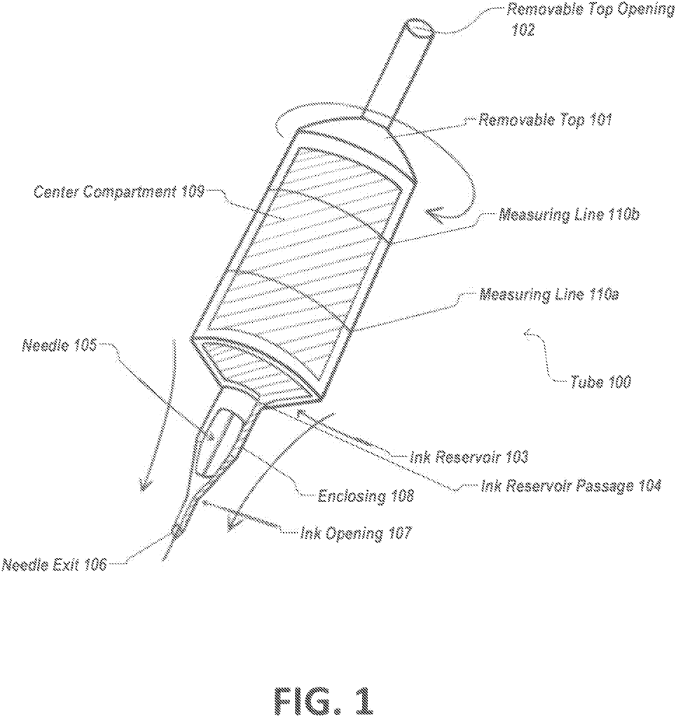

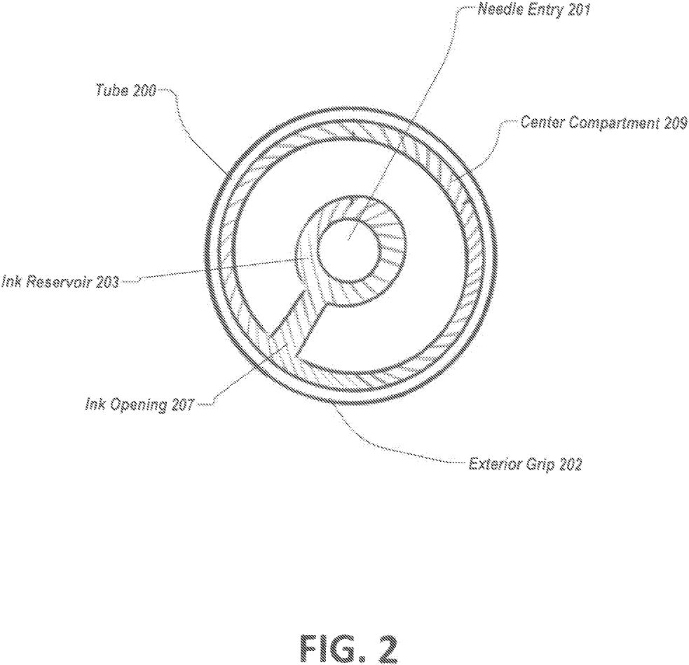

FIG. 1 illustrates a perspective view of a tattoo ink dispenser, in accordance with some embodiments of the present disclosure. As illustrated, the tattoo ink dispenser can include a tube 100 that has a removable top 101 with a removable top opening 102, an ink reservoir 103 with an ink reservoir passage 104, a needle 105, a needle exit 106, an ink opening 107, an enclosing 108, and a center compartment 109 with measuring lines 110a and 110b.

In some embodiments, the tube 100 of the tattoo ink dispenser can be constructed of stainless steel material. In some embodiments, the tube 100 can be constructed of plastic. In some embodiments, the tube 100 can be constructed of other materials appropriate for tattoo machines, including, but not limited to, copper, aluminum, etc.

In some embodiments, the removable top 101 of the tattoo ink dispenser can be connected to one end of the tube 100 (e.g., the top surface of the tube 100). In some embodiments, the removable top 101 can be constructed of the same or a different material as the tube 100. For example, the removable top 101 can be constructed of materials including, but not limited to, stainless steel, plastic, copper, aluminum, etc. In some embodiments, the removable top 101 can be circular or cylindrical in shape. In some embodiments, the removable top 101 can be shaped to fit the shape of the top surface of the tube 100, i.e., the removable top 101 can be shaped to enclose the top surface of the tube 100. In some embodiments, the removable top 101 enables a user (e.g., a tattoo artist) to remove the removable top 101 in order to fill the center compartment 109 of the tattoo ink dispenser with ink. In some embodiments, removing the removable top 101 can include, for example, twisting, screwing, and/or turning the removable top 101 in a circular motion (e.g., clockwise or counterclockwise) in order to disconnect the removable top 101 from the top surface of the tube 100. In some embodiments, removing the removable top 101 can include vertically lifting the removable top 101 off the top surface of the tube 100 in order to disconnect the removable top from the top surface of the tube 100. In some embodiments, the removable top 101 may be fitted with a gasket to ensure a tight seal between the removable top 101 and the tube 100. In some embodiments, the gasket may be made of a malleable, water-resistant material such as rubber or silicone. In all embodiments, the removable top 101 ensures that the ink filled inside the tube 100 is tightly sealed and not leaking.

In some embodiments, the removable top 101 is hollow or partially hollow (and partially solid) and has a removable top opening 102. In some embodiments, the removable top opening 102 allows a needle 105 to move in a vertical direction throughout the tattoo ink dispenser and further allows the needle to be attached to a tattoo machine. In some embodiments, the needle 105 can be located in the middle of the removable top opening 102 and can be equidistant from each side of the removable top opening 102.

In some embodiments, the center compartment 109 of the tattoo ink dispenser can be a hollow structure at the center of the tube 100. In some embodiments, the center compartment 109 can be marked with measuring lines 110a and 110b, such that the tattoo artist can use the measuring lines 110a and 110b to measure a particular amount of ink to be filled in the center compartment 109. The measuring lines 110a and 110b on the center compartment 109 can be one or more lines drawn on the exterior surface of the center compartment 109 to indicate a unit of measurement. The unit of measurement indicated by measuring lines 110a and 110b are related to the internal volume of the center compartment measured at each of the measuring line 110a and 110b.

In some embodiments, the ink reservoir 103 can be a compartment, where the ink reservoir 103 is connected to a bottom portion of the center compartment 109. A bottom portion of the ink reservoir 103 can be connected to a portion of an outer surface of the tube (e.g., an enclosing 108) via an ink reservoir passage 104. The enclosing 108 may contain a needle 105. The ink reservoir 103 can extend alongside the enclosing 108 towards a bottom portion of the enclosing 108 and/or the tip of the needle 105. In some embodiments, the bottom portion of the ink reservoir 103 (i.e., the end of the reservoir closest to the tip of the needle 105) can include an ink opening 107 (e.g., a small hole or opening). In some embodiments, once a tattoo artist fills the center compartment 109 of the tattoo ink dispenser with ink, the ink can flow downward through the ink reservoir 103 and through the ink opening 107. The ink can then adhere onto the needle 105 for use in the tattooing process.

In some embodiments, the needle 105 of the tattoo ink dispenser can be located within the enclosing 108. In some embodiments, the needle 105 can be located in the middle of the enclosing 108 and can be equidistant from each side of the enclosing 108. The tip of the needle 105 can extend beyond the bottom portion of the enclosing 108 through the needle exit 106. In some embodiments, the top portion of the enclosing 108 can be connected to the bottom portion of the ink reservoir 103 and/or the center compartment 109. In some embodiments, the enclosing 108 can be constructed of the same material as the tube 100. In some embodiments, the enclosing 108 can be constructed of a different material than the tube 100. For example, the enclosing 108 can be constructed of stainless steel, plastic, copper, aluminum, etc.

FIG. 2 illustrates an aerial top view of the tattoo ink dispenser, in accordance with some embodiments of the present disclosure. As illustrated, the tattoo ink dispenser can include a tube 200 (e.g., the tube 100 of FIG. 1) surrounded by an outer surface (e.g., an exterior grip 202). In some embodiments, the tattoo ink dispenser can include a center compartment 209 (e.g., the center compartment 109 of FIG. 1) within the tube 200. The center compartment 209 can contain ink that the tattoo artist fills in by removing a removable top (e.g., the removable top 101 of FIG. 1), as described herein above with reference to FIG. 1.

In some embodiments, the exterior grip 202 can be constructed of the same material as the tube 200. In some embodiments, the exterior grip can be constructed of a different material than the tube 200. For example, the exterior grip 202 can be constructed of rubber, stainless steel, plastic, copper, aluminum, etc. In some embodiments, the exterior grip 202 can allow for tattoo artists to hold the tattoo ink dispenser for use during the tattooing process.

In some embodiments, the tattoo ink dispenser can include a needle entry 201 to insert a needle (e.g., the needle 105 of FIG. 1) into an enclosing (e.g., the enclosing 108 of FIG. 1) of the tattoo ink dispenser, as described herein above with reference to FIG. 1. A tattoo artist can insert the needle through the needle entry 201 at the bottom portion of the enclosing (e.g., the tip of the tube). In some embodiments, needles of various sizes, lengths, and materials can be inserted into the tattoo ink dispenser.

In some embodiments, the center compartment 209 can feed the ink into the ink reservoir 203 (e.g., the ink reservoir 103 of FIG. 1) through the ink opening 207 (e.g., the ink reservoir opening 107 of FIG. 1), as described herein above with reference to FIG. 1.

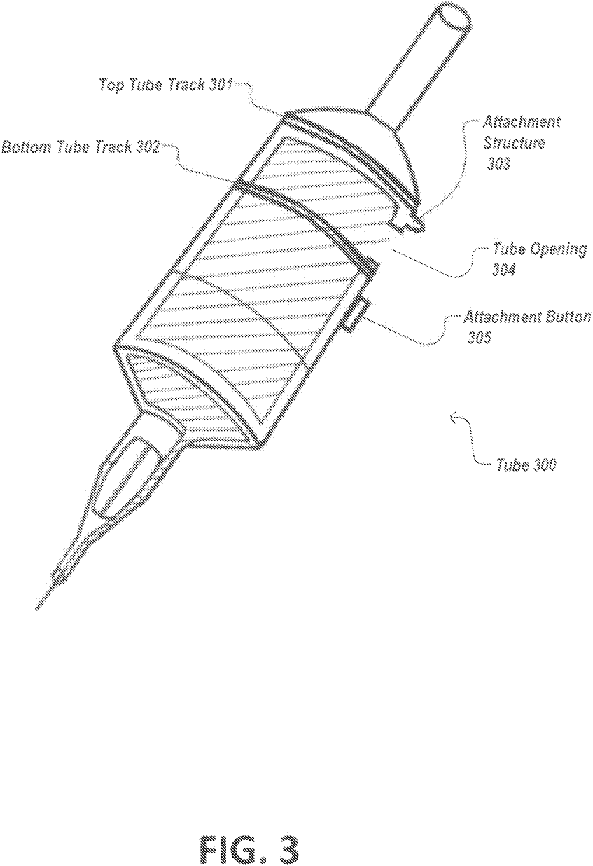

FIG. 3 illustrates a perspective view of an tattoo ink dispenser that can dispense different colors of ink stored in removable cartridges that can be fitted to the dispenser, in accordance with some embodiments of the present disclosure. As illustrated, the interchangeable tattoo ink dispenser can include a tube 300 (e.g., the tube 100 of FIG. 1) that has a top tube track 301 and a bottom tube track 302, an attachment structure 303, a tube opening 304, and an attachment button 305.

In some embodiments, the top tube track 301 and the bottom tube track 302 each form a channel on which a removable tattoo ink cartridge may be affixed. In some embodiments, the top tube track 301 and the bottom tube track 302 are built into the tube 300. In some embodiments, this is done be engraving or carving out the channel for the top tube track 301 and the bottom tube track 302. In some embodiments, this is done by a molding process. In some embodiments, the top tube track 301 and the bottom tube track 302 are the same material as the tube 300. In some embodiments, the top tube track 301 and the bottom tube track 302 can be constructed of a different material than the tube 300. For example, the top tube track 301 and the bottom tube track 302 can be constructed of stainless steel, plastic, copper, aluminum, etc.

In some embodiments, the attachment structure 303 may be a projection from the tube 300. In some embodiments, the attachment structure 303 may be constructed of the same material as the tube 300. In some embodiments, the attachment structure 303 may be constructed of a different material than the tube 300. For example, the attachment structure 303 may be constructed of stainless steel, plastic, copper, aluminum, etc. In some embodiments, the attachment structure 303 may be retractable such that when retracted, it recesses into the tube 300 and becomes flush with the surface of the tube 300. In some embodiments, the attachment structure 303 may be retracted by the attachment button 305. The attachment button 305 may retract the attachment structure 303 when it is depressed, and may allow the attachment structure 303 to return to its original non-recessed position when it is not depressed. The attachment button 305 may also function as a slide to retract attachment structure 303 when slid in a first direction and return the attachment structure 303 to its original non-recessed position when it is slid in a direction opposite the first direction. In some embodiments, the attachment button 305 may be constructed of the same material as the tube 300. In some embodiments, the attachment button 305 may be constructed of a different material than the tube 300. For example, the attachment button 305 may be constructed of stainless steel, plastic, copper, aluminum, etc.

In some embodiments, the tube 300 may have a tube opening 304 which provides an opening (e.g., passage or channel) to the tube 300 and into a central compartment (e.g., the central compartment 109 of FIG. 1).

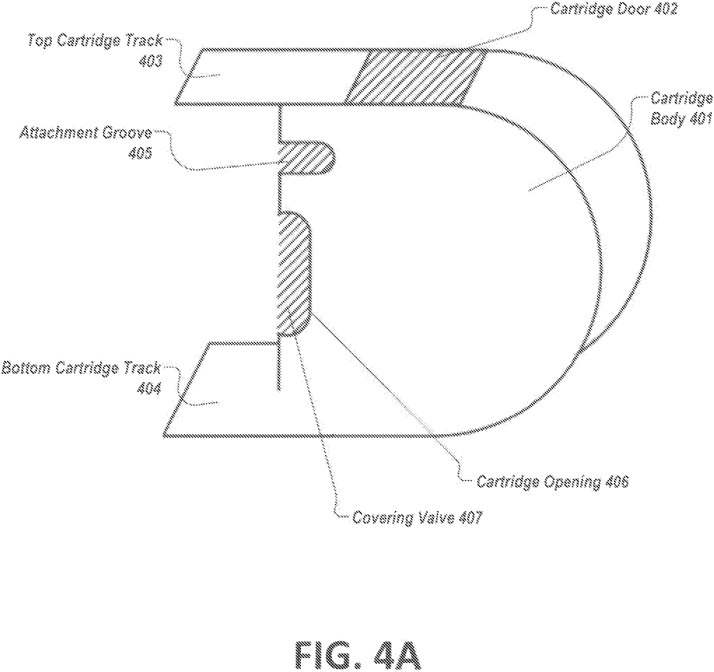

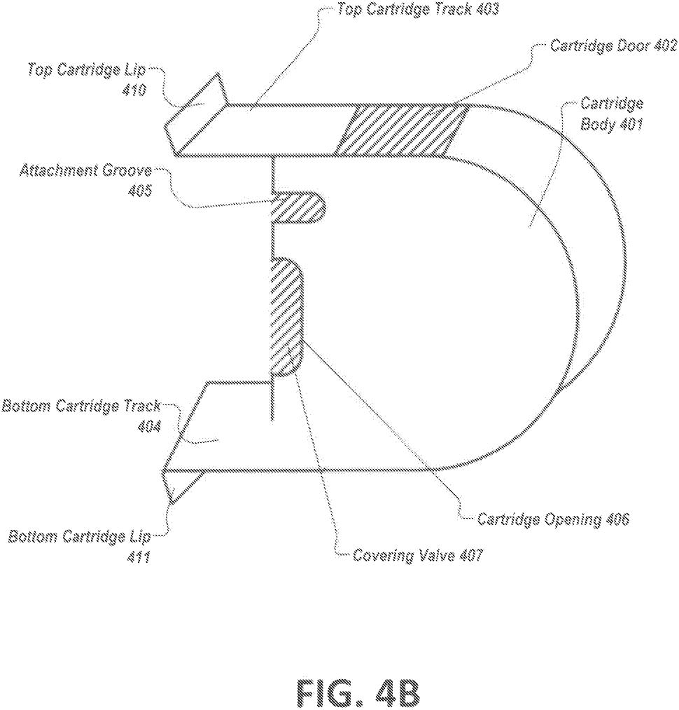

FIGS. 4A-B illustrates a perspective view of a removable tattoo ink cartridge, in accordance with some embodiments of the present disclosure. As illustrated, the removable tattoo ink cartridge can include a cartridge body 401, a cartridge door 402, a top cartridge track 403, a bottom cartridge track 404, an attachment groove 405, a cartridge opening 406, and a covering valve 407. In some embodiments, the removable tattoo ink cartridge may be constructed of the same material as a tube (e.g., the tube 100 of FIG. 1). In some embodiments, the removable tattoo ink cartridge may be constructed of a different material than the tube 100. For example, the removable tattoo ink cartridge may be constructed of stainless steel, plastic, copper, aluminum, etc.

In some embodiments, the cartridge body 401 may be hollow and is configured to store tattoo pigment or ink. In some embodiments, the cartridge opening 406 may create a channel to the interior of the cartridge body 401. In some embodiments, the cartridge opening is configured to align with a tube opening (e.g., the tube opening 304 of FIG. 3). In some embodiments, the cartridge opening 406 is fitted with a covering valve 407. In some embodiments, the covering valve 407 may be in a closed position when the attachment groove 405 is empty and in an opened position when the attachment groove 405 is filled with an attachment structure (e.g., the attachment structure 303 of FIG. 3).

In some embodiments, the cartridge door 402 may be opened to the cartridge body. The cartridge door 402 may be opened in order to receive and store tattoo pigment or ink. In some embodiments, the top cartridge track 403 is configured to align with a top tube track (e.g., the top tube track 301 of FIG. 3) and the bottom cartridge track 404 is configured to align with a bottom tube track (e.g., the bottom tube track 302 of FIG. 3). In some embodiments, the top cartridge track 403 has a top cartridge lip 410. In some embodiments, the bottom cartridge track 404 has a bottom cartridge lip 411. In some embodiments, the top cartridge lip 410 is configured to fit within the top tube track and the bottom cartridge lip 411 is configured to fit within the bottom tube track.

In some embodiments, the terms “first,” “second,” “third,” “fourth,” etc. as used herein are meant as labels to distinguish among different elements and do not have an ordinal meaning according to their numerical designation.

The above description is intended to be illustrative, and not restrictive. Although the present disclosure has been described with references to specific illustrative examples and implementations, it will be recognized that the present disclosure is not limited to the examples and implementations described. The scope of the disclosure should be determined with reference to the following claims, along with the full scope of equivalents to which the claims are entitled.

The preceding description sets forth numerous specific details such as examples of specific systems, components, methods, and so forth in order to provide a good understanding of several embodiments of the present disclosure. It will be apparent to one skilled in the art, however, that at least some embodiments of the present disclosure may be practiced without these specific details. In other instances, well-known components or methods are not described in detail to avoid unnecessarily obscuring the present disclosure. Thus, the specific details set forth are merely exemplary. Particular implementations may vary from these exemplary details and still be contemplated to be within the scope of the present disclosure.

The terms “over,” “under,” “between,” “disposed on,” and “on” as used herein refer to a relative position of one material layer or component with respect to other layers or components. For example, one layer disposed on, over, or under another layer may be directly in contact with the other layer or may have one or more intervening layers. Moreover, one layer disposed between two layers may be directly in contact with the two layers or may have one or more intervening layers. Similarly, unless explicitly stated otherwise, one feature disposed between two features may be in direct contact with the adjacent features or may have one or more intervening layers.

The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion.

Reference throughout this specification to “one embodiment,” “an embodiment,” or “some embodiments” means that a particular feature, structure, or characteristic described in connection with the embodiment is included in at least one embodiment. Thus, the appearances of the phrase “in one embodiment,” “in an embodiment,” or “in some embodiments” in various places throughout this specification are not necessarily all referring to the same embodiment. In addition, the term “or” is intended to mean an inclusive “or” rather than an exclusive “or.” That is, unless specified otherwise, or clear from context, “X includes A or B” is intended to mean any of the natural inclusive permutations. That is, if X includes A, X includes B, or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. When the term “about,” “substantially,” or “approximately” is used herein, this is intended to mean that the nominal value presented is precise within ± 10%.

It is understood that the above description is intended to be illustrative, and not restrictive. Many other embodiments will be apparent to those of skill in the art upon reaching and understanding the above description. The scope of the disclosure should, therefore, be determined with reference to the appended claims, along with the full scope of equivalents to which such claims are entitles.

What is claimed is:

1. A tattoo ink dispenser apparatus comprising:
a tube, wherein the tube comprises at least a removable top and a center compartment, and wherein the center compartment is a hollow structure configured to store ink; and
a bottom portion of the tube with a central opening to place and hold a needle therein, wherein the stored ink flows downwards and adheres to the needle to create a tattoo on a surface.

2. The tattoo ink dispenser apparatus of claim 1, wherein the bottom portion of the tube comprises:
an ink reservoir;
an enclosing;
an ink reservoir passage connecting the ink reservoir and the enclosing;
a needle exit; and
an ink opening connecting the enclosing and the needle exit;
wherein the needle passes through the central opening of the bottom portion of the tube and through the needle exit.

3. The tattoo ink dispenser apparatus of claim 2, wherein the needle moves in a substantially vertical motion from a first position to a second position when operatively coupled to a tattoo machine.

4. The tattoo ink dispenser apparatus of claim 3, wherein the ink opening is at a position substantially similar to the second position.

5. The tattoo ink dispenser apparatus of claim 2, wherein the center compartment is open to the ink reservoir wherein the stored ink flows downward from the center compartment to the ink reservoir.

6. The tattoo ink dispenser apparatus of claim 2, wherein the enclosing substantially surrounds the needle.

7. The tattoo ink dispenser apparatus of claim 1, wherein an exterior grip substantially surrounds the tube.

8. The tattoo ink dispenser apparatus of claim 1, wherein the tube further comprises a plurality of measuring lines.

9. The tattoo ink dispenser apparatus of claim 8, wherein the plurality of measuring lines are indicative of an internal volume of the center compartment.

10. The tattoo ink dispenser apparatus of claim 1, wherein the removable top is hollow and has a removable top opening configured to substantially align with the central opening, and wherein the needle extends through the removable top opening.

11. The tattoo ink dispenser apparatus of claim 1, wherein the removable top has a gasket configured to prevent a leak of the stored ink.

12. A system comprising:
an tattoo ink dispenser with interchangeable ink, the tattoo ink dispenser comprising:
a tube, wherein the tube comprises at least a removable top and a center compartment, and wherein the center compartment is a hollow structure configured to store ink; and
a bottom portion of the tube with a central opening to place and hold a needle therein, wherein the stored ink flows downwards and adheres to the needle to create a tattoo on a surface; and
a removable tattoo ink cartridge comprising:
a cartridge body, wherein the cartridge body is hollow and is configured to store ink and to be operatively coupled with the tube by a top cartridge track, a bottom cartridge track, an attachment groove, and a cartridge opening.

13. The system of claim 12, wherein the tube further comprises:
a top tube track, configured to align with the top cartridge track;
a bottom tube track, configured to align with the bottom cartridge track;
an attachment structure, configured to align with the attachment groove;
a tube opening, configured to align with the cartridge opening; and
an attachment button.

14. The system of claim 13, wherein responsive to the attachment structure being operatively coupled with the attachment groove, a covering valve moves from a first position to a second position, wherein the second position is configured to couple the tube opening with the cartridge opening.

15. The system of claim 13, wherein the attachment button is operatively coupled with the attachment structure such that when the attachment button is moved from a first position to a second position, the attachment structure moves from a third position to a fourth position, wherein the third position is projecting from the tube and the fourth position is retracted into the tube.

16. The system of claim 12, wherein the removable ink cartridge further comprise a cartridge door configured to open to the cartridge body.

17. The system of claim 12, wherein a first removable tattoo ink cartridge stores a first color of ink and a second removable tattoo ink cartridge stores a second color of ink.

18. The system of claim 17, wherein the second removable tattoo ink cartridge is fitted with the tube after removing the first removable tattoo ink cartridge that was previously fitted with the tube.
